(19) United States
(12) Patent Application Publication
(10) Pub. No.: US 2010/0076262 A1
(43) Pub. Date: Mar. 25, 2010

(54) ENDOSCOPE INSPECTION SYSTEM

(75) Inventors: Wei-Zen Sun, Taipei City (TW); Jaw Fu-Shan, Taipei City (TW)

Correspondence Address: QUINTERO LAW OFFICE, PC, 615 Hampton Dr, Suite A202, Venice, CA, 90291, US

(73) Assignee: NATIONAL TAIWAN UNIVERSITY, Taipei (TW)

(21) Appl. No.: 12/562,948

(22) Filed: Sep. 18, 2009

(30) Foreign Application Priority Data
Sep. 19, 2008 (TW) TW097136047

(51) Int. Cl. A61B 1/04

(52) U.S. Cl. 600/109

(57) ABSTRACT

An endoscope inspection system. An image-capturing device is disposed on an endoscope probe and is electrically connected to a signal conditioning unit. A differential electrode set includes an annular detecting electrode and a reference electrode. The annular detecting electrode surrounds the endoscope probe. The annular detecting electrode, reference electrode, and display are electrically connected to the signal conditioning unit.

### CROSS REFERENCE TO RELATED APPLICATIONS

This Application claims priority of Taiwan Patent Application No. 097136047, filed on Sep. 19, 2008, the entirety of which is incorporated by reference herein.

### BACKGROUND OF THE INVENTION

1. Field of the Invention

The invention relates to an endoscope inspection system, and more particularly to an endoscope inspection system synchronously detecting vital signs.

2. Description of the Related Art

An endoscope is one of the commonly used medical instruments in medical institutions. For a small or remote medical institution, a multi-function endoscope can help reduce laborer costs and provide better medical services. When performing an endoscope inspection, medical personnel must properly adjust the progress of the inspection or even halt the inspection according to a subject's response. Clinically, the subject may feel uncomfortable when the medical personnel are looking for a nidus. Specifically, a juvenile or paralyzed subject may not be immediately express uncomfortable feelings. Thus, the endoscope inspection must be performed with objective physiological parameters, timely responding to the physiological conditions of the subject. For example, when the subject feels pain or physiological functions of the subject descend, a heartbeat rate and a respiratory rate thereof may be promptly responsive.

Accordingly, the heartbeat and respiratory rates are critical vital signs and can be respectively detected by an electrocardiograph and a thoracic elastic ring. Nevertheless, as sensors for detecting the heartbeat and respiratory rates are different, it is not easy to simultaneously detect the heartbeat and respiratory rates.

Hence, there is a need for an endoscope inspection system capable of synchronously detecting a heartbeat rate and a respiratory rate, promoting quality of medical inspections.

### BRIEF SUMMARY OF THE INVENTION

A detailed description is given in the following embodiments with reference to the accompanying drawings.

An exemplary embodiment of the invention provides an endoscope inspection system for synchronously detecting vital signs during an endoscope inspection. The endoscope inspection system comprises a signal conditioning unit, an endoscope probe, an image-capturing device, a differential electrode set, and a display. The image-capturing device is disposed on the endoscope probe and is electrically connected to the signal conditioning unit. The differential electrode set comprises an annular detecting electrode and a reference electrode. The annular detecting electrode surrounds the endoscope probe. The annular detecting electrode, reference electrode, and display are electrically connected to the signal conditioning unit.

The endoscope inspection system further comprises a power supply device electrically connected to the signal conditioning unit, providing the signal conditioning unit and image-capturing device with electric power.

The power supply device comprises a cell.

The surface area of the reference electrode exceeds that of the annular detecting electrode.

The ratio of the surface area of the reference electrode to that of the annular detecting electrode exceeds 10.

The signal conditioning unit comprises a high-pass filter, an instrument amplifier, a gain stage amplifier, a low-pass filter, and a digital band pass filter, which are all sequentially and electrically connected to each other.

### BRIEF DESCRIPTION OF THE DRAWINGS

The invention can be more fully understood by reading the subsequent detailed description and examples with references made to the accompanying drawings, wherein:

FIG. 1 is a schematic perspective view of an endoscope inspection system.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

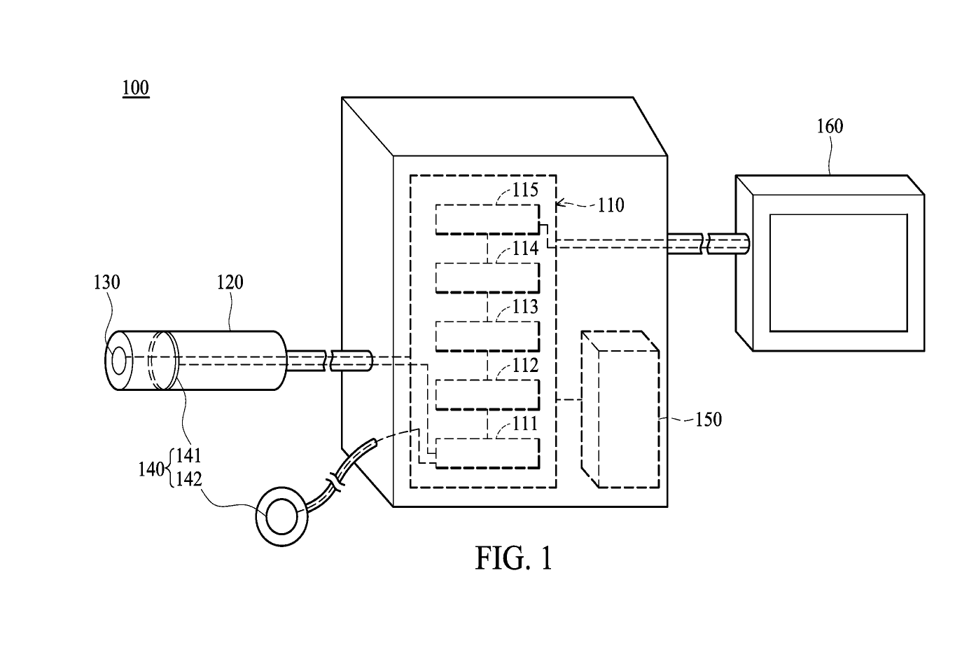

Referring to FIG. 1, an endoscope inspection system 100 comprises a signal conditioning unit 110, an endoscope probe 120, an image-capturing device 130, a differential electrode set 140, a power supply device 150, and a display 160.

The signal conditioning unit 110 comprises a high-pass filter 111, an instrument amplifier 112, a gain stage amplifier 113, a low-pass filter 114, and a digital band pass filter 115, which are all sequentially and electrically connected to each other.

The image-capturing device 130 is disposed on (a front end of) the endoscope probe 120 and is electrically connected to the signal conditioning unit 110. In this embodiment, the image-capturing device 130 may be a complementary metal oxide semiconductor (CMOS), receiving images.

The differential electrode set 140 comprises an annular detecting electrode 141 and a reference electrode 142. In this embodiment, the annular detecting electrode 141 surrounds (the outer surface of) the endoscope probe 120, and the annular detecting electrode 141 and reference electrode 142 are electrically connected to the signal conditioning unit 110. Specifically, the annular detecting electrode 141 and reference electrode 142 are electrically connected to the high-pass filter 111 of the signal conditioning unit 110. Moreover, when the surface area of the annular detecting electrode 141 does not match that of the reference electrode 142, the strength of detected and received signals can be enhanced. Thus, in this embodiment, the surface area of the reference electrode 142 exceeds that of the annular detecting electrode 141. Preferably, the ratio of the surface area of the reference electrode 142 to that of the annular detecting electrode 141 exceeds 10.

The power supply device 150 is electrically connected to the signal conditioning unit 110, providing the signal conditioning unit 110 and image-capturing device 130 with electric power. Additionally, in this embodiment, the power supply device 150 may be a cell.

The display 160 is electrically connected to the signal conditioning unit 110.

The following description is directed to an endoscope inspection process and operation of synchronously detecting a heartbeat rate and a respiratory rate using the endoscope inspection system 100.

Medical personnel can attach the reference electrode 142 of the differential electrode set 140 to the body surface of a subject and insert the endoscope probe 120 into the body cavity thereof. Here, the image-capturing device 130 disposed on (the front end of) the endoscope probe 120 can receive images from the interior of the body cavity and transmit the same to the display 160 through the signal conditioning unit 110 for inspection or examination. At the same time, the annular detecting electrode 141 surrounding (the outer surface of) the endoscope probe 120 and the reference electrode 142 attached to the body surface of the subject can detect a physiological signal. Specifically, as the annular detecting electrode 141 surrounds (the outer surface of) the endoscope probe 120, contact between the annular detecting electrode 141 and the body cavity is not limited to a specific direction or plane, thereby facilitating signal detection.

The physiological signal detected by the differential electrode set 140 (annular detecting electrode 141 and reference electrode 142) is transmitted to the high-pass filter 111 of the signal conditioning unit 110. The high-pass filter 111 then removes extremely low frequency noises from the physiological signal. Here, even though the extremely low frequency noises are removed from the physiological signal, enormous common mode noises exist therein. Thus, the physiological signal is further transmitted to the instrument amplifier 112 providing a high common mode rejection ratio (CMRR) and the common mode noises are removed thereby. Then, the physiological signal is transmitted to the gain stage amplifier 113 and is amplified thereby. The amplified physiological signal is then transmitted to the low-pass filter 114 and the low-pass filter 114 removes extremely high frequency noises therefrom. At this point, the physiological signal is converted into an electro-cardio signal including the heartbeat and respiratory rates. Here, because the heartbeat and respiratory rates are respectively referred to as a high-frequency signal (about 1 Hz to 10 Hz) and a low-frequency signal (about 0.1 Hz to 0.2 Hz), discovery of the respiratory rate is difficult. Accordingly, the electro-cardio signal is further transmitted to the digital band pass filter 115 and the heartbeat rate is separated from the respiratory rate thereby. The separated heartbeat and respiratory rates and the images received by the image-capturing device 130 are then transmitted to the display 160 and are displayed thereby.

Accordingly, the medical personnel can simultaneously monitor the vital signs of the subject (i.e. the medical personnel can synchronously detect the heartbeat and respiratory rates of the subject) when using the endoscope inspection system 100 to perform the endoscope inspection, thereby easily and objectively estimating the physiological condition of the subject. Thus, the endoscope inspection system 100 can enhance convenience and quality of the medical inspection. Additionally, as the endoscope inspection system 100 can synchronously detect the heartbeat and respiratory rates during the endoscope inspection, labor and equipment costs required by a medical institution can be reduced.

Alternatively, the display 160 may be connected to the signal conditioning unit 110 with a wireless transmission manner, simplifying arrangement of power lines.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An endoscope inspection system, comprising:
a signal conditioning unit;
an endoscope probe;
an image-capturing device disposed on the endoscope probe and electrically connected to the signal conditioning unit;
a differential electrode set comprising an annular detecting electrode and a reference electrode, wherein the annular detecting electrode surrounds the endoscope probe, and the annular detecting electrode and reference electrode are electrically connected to the signal conditioning unit; and
a display electrically connected to the signal conditioning unit.

2. The endoscope inspection system as claimed in claim 1, further comprising a power supply device electrically connected to the signal conditioning unit, providing the signal conditioning unit and image-capturing device with electric power.

3. The endoscope inspection system as claimed in claim 2, wherein the power supply device comprises a cell.

4. The endoscope inspection system as claimed in claim 1, wherein the surface area of the reference electrode exceeds that of the annular detecting electrode.

5. The endoscope inspection system as claimed in claim 1, wherein the ratio of the surface area of the reference electrode to that of the annular detecting electrode exceeds 10.

6. The endoscope inspection system as claimed in claim 1, wherein the signal conditioning unit comprises a high-pass filter, an instrument amplifier, a gain stage amplifier, a low-pass filter, and a digital band pass filter, which are all sequentially and electrically connected to each other.
